(19) United States
(12) Patent Application Publication
(10) Pub. No.: US 2026/0226092 A1
(43) Pub. Date: Aug. 6, 2026

(54) COCRYSTALS OF CORTEXOLONE 17-PROPIONATE AND METHODS FOR THEIR PREPARATION

(71) Applicant: Farmabios S.p.A., Gropello Cairoli (PV) (IT)

(72) Inventors: Mauro GABOARDI, Novara (NO) (IT); Mario DI GIACOMO, Cenate Sotto (BG) (IT); Nicolas TESSON, Barcelona (ES); Lydia CÁRDENAS ROMAÑA,, Barcelona (ES); Montserrat TRILLA CASTANO, Barcelona (ES)

(21) Appl. No.: 19/147,899

(22) PCT Filed: Feb. 5, 2024
(86) PCT No.: PCT/EP2024/052793

§ 371 Date: Jul. 14, 2025

(30) Foreign Application Priority Data
Jul. 2, 2023 (IT) 102023000001929

(51) Int. Cl. C07J 5/00; A61K 31/573; C07C 39/07; C07C 39/08; C07C 65/10

(52) U.S. Cl. CPC C07J 5/0053; A61K 31/573; C07C 39/07; C07C 39/08; C07C 65/10; C07B 2200/13

(57) ABSTRACT

Cocrystals of cortexolone 17α-propionate with some coformers are provided, as well as methods for their preparation.

The present invention is directed to cocrystals of cortexolone 17α-propionate with some coformers, as well as methods for their preparation.

Cortexolone 17α-propionate is a steroidal antiandrogen used for the topical treatment of androgen-dependent skin diseases, such as acne, hirsutism or androgenetic alopecia.

The active ingredient cortexolone 17α-propionate, also known as clascoterone or 17α, 21-dihydroxypregn-4-en-3,20-dione 17α-propionate, has the following formula (I):

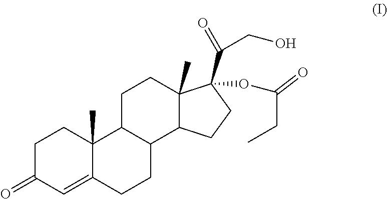 [SMILES: CCC(=O)O[C@]1(C(=O)CO)CCC2C3CCC4=CC(=O)CC[C@]4(C)C3CC[C@@]21C]

and is marketed as a medicinal specialty in the form of cream, gel or ointment for topical application, under the brand name Winlevi®.

Several crystalline forms of cortexolone 17α-propionate are known.

The patent application WO 2009/019138 describes three anhydrous crystalline forms I, II and III and a hydrated form IV.

The article Steroids 2017, 128, 95-104 reports the characterization of two pseudopolymorphs CPIII and CPW, respectively anhydrous and hydrated forms of cortexolone 17α-propionate. These two pseudopolymorphs correspond to forms III and IV described in the patent application WO 2009/019138.

The patent application IT 102021000008429 describes a new hydrated form obtained by crystallization of cortexolone 17α-propionate in a mixture of two organic solvents.

However, the known crystalline forms have several disadvantages that limit their use in the preparation of a pharmaceutical form, such as the conversion of anhydrous forms to hydrated forms during formulation.

We have now found that the formation of cocrystals between cortexolone 17α-propionate and specific coformers allows to overcome the disadvantages of the prior art and to improve some physico-chemical properties of the product, such as hygroscopicity, apparent solubility, dissolution speed, flowability, chemical stability.

For example, the cocrystal of Berberine hydrochloride with citric acid shows less hygroscopicity than the corresponding hydrochloride salt (International Journal of Pharmaceutics, 2019, 554, 14-20).

Other examples of cocrystals demonstrate that it is possible to have different dissolution profiles, which can have advantages in terms of releasing the formulated product, for example Fluoxetine HCl cocrystals (S. L. Childs et al. JACS, 2004, 13335).

A final aspect could concern the increase in permeability of the cocrystal with respect to the active ingredient. Carbamazepine cocrystals with vanillic or succinic acid increase permeability in human intestinal cells (Mol. Pharmaceutics, 2018, 268).

### BRIEF DESCRIPTION OF THE FIGURES

FIG. 1: XRPD spectrum of crystalline form B (cocrystal with salicylic acid)

FIG. 2: DSC thermogram of crystalline form B

FIG. 3: ¹H NMR spectrum of crystalline form B

FIG. 4: XRPD spectrum of crystalline form D (cocrystal with resorcinol)

FIG. 5: DSC thermogram of crystalline form D

FIG. 6: ¹H NMR spectrum of crystalline form D

FIG. 7: XRPD spectrum of crystalline form F (cocrystal with m-cresol)

FIG. 8: DSC thermogram of crystalline form F

FIG. 9: ¹H NMR spectrum of crystalline form F

FIG. 10: XRPD spectrum of crystalline form G (cocrystal with m-cresol)

FIG. 11: DSC thermogram of crystalline form G

FIG. 12: ¹H NMR spectrum of crystalline form G

FIG. 13: XRPD spectrum of crystalline form H (cocrystal with 3,4-xylenol)

FIG. 14: DSC thermogram of crystalline form H

FIG. 15: ¹H NMR spectrum of crystalline form H

FIG. 16: XRPD spectrum of crystalline form I (cocrystal with o-cresol)

FIG. 17: DSC thermogram of crystalline form I

FIG. 18: ¹H NMR spectrum of crystalline form I

FIG. 19: XRPD spectrum of crystalline form J (cocrystal with resorcinol)

FIG. 20: DSC thermogram of crystalline form J

FIG. 21: ¹H NMR spectrum of crystalline form J

FIG. 22: Solubility profile of crystalline form J, at pH 5.1 (dotted line) and at pH 6.0 (continuous line)

FIG. 23: Solubility profile of crystalline form G, at pH 5.1 (dotted line) and at pH 6.0 (continuous line)

FIG. 24: Solubility profile of crystalline form III, at pH 5.1 (dotted line) and at pH 6.0 (continuous line)

FIG. 25: SEM photographs of crystalline form J at different magnifications: (A) at 504X, (B) at 1.02 KX, (C) at 2.13 KX and (D) at 4.09 KX

FIG. 26: SEM photographs of crystalline form G at different magnifications: (A) at 504X, (B) at 1.05 KX and (C) at 2.02 KX

FIG. 27: SEM photographs of crystalline form III at different magnifications: (A) at 504X, (B) at 1.02 KX and (C) at 2.05 KX

The object of the present invention is therefore a cocrystal between cortexolone 17α-propionate and a coformer.

Examples of such coformers that can be used in the present invention are substituted aromatic compounds, such as resorcinol, m-cresol, o-cresol, 3,4-xylenol, salicylic acid, gentisic acid, benzyl alcohol, benzoic acid, 2,4-dihydroxybenzoic acid.

We have in fact discovered that cortexolone 17α-propionate with the aforementioned coformers is capable of forming non-hygroscopic cocrystals.

According to a preferred embodiment, said cocrystal comprises cortexolone 17α-propionate and salicylic acid in a 2:1 molar ratio in crystalline form B.

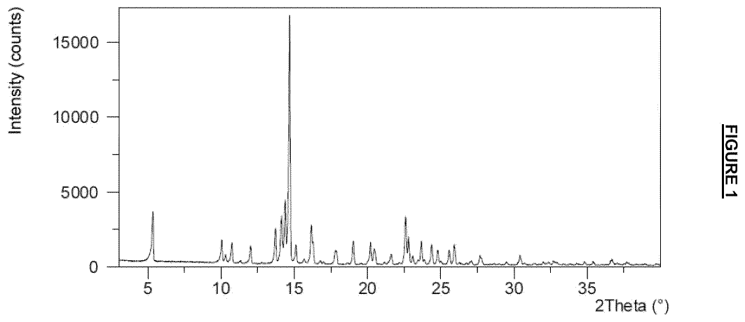

More preferably, such crystalline form B is characterized by an XRPD spectrum having the following characteristic peaks at approximately 5.4; 13.7; 14.1; 14.4; 14.7; 16.2; 22.6±0.2° 2θ or as shown in FIG. 1.

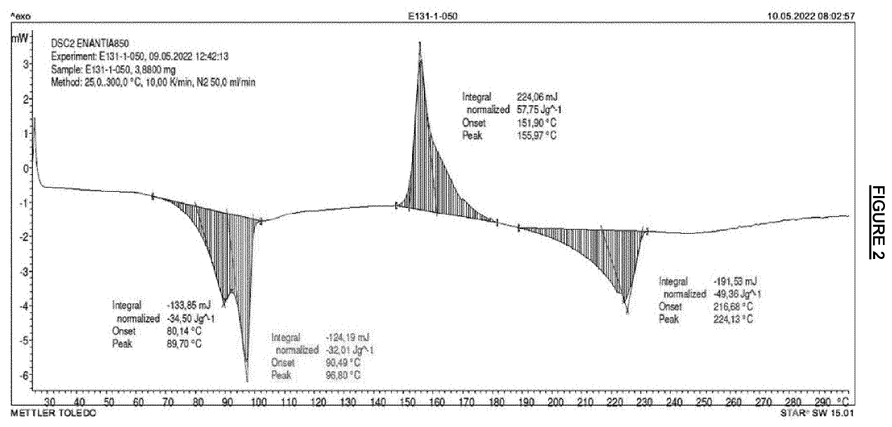

According to a preferred embodiment, said crystalline form B has a DSC thermogram as shown in FIG. 2.

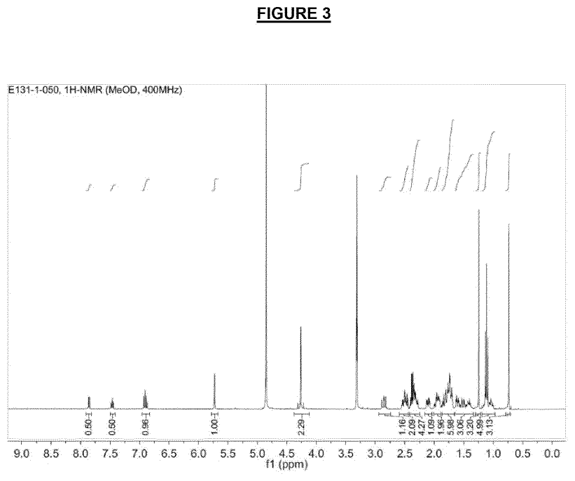

According to a preferred embodiment, said crystalline form B has a proton NMR spectrum as shown in FIG. 3.

Preferably, this crystalline form B is obtained by suspending cortexolone 17α-propionate in crystalline form III with salicylic acid in a 2:1 molar ratio in methyl-t-butyl ether.

According to a further preferred embodiment, said cocrystal comprises cortexolone 17α-propionate and resorcinol in a 1:1 molar ratio in crystalline form D.

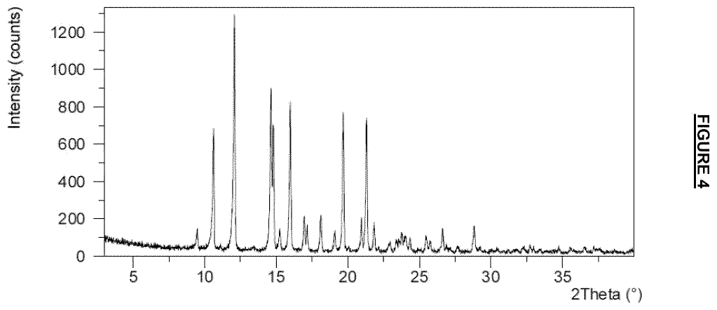

More preferably, such crystalline form D is characterized by an XRPD spectrum having the following characteristic peaks at approximately 10.6; 12.1; 14.6; 14.8; 16.0; 19.7; 21.3±0.2° 2θ or as shown in FIG. 4.

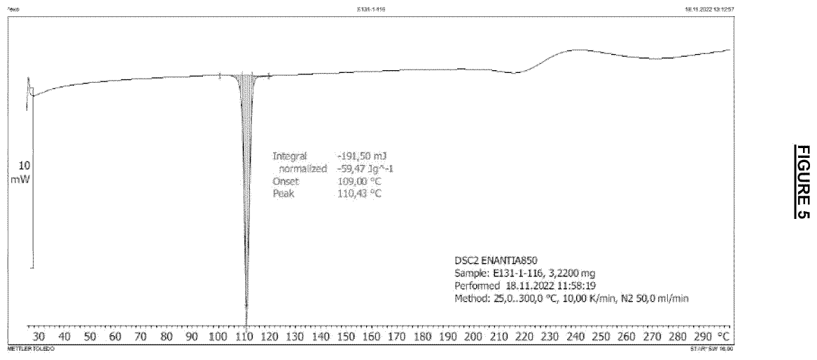

According to a preferred embodiment, said crystalline form D has a melting point at approximately 109° C. and a DSC thermogram as shown in FIG. 5.

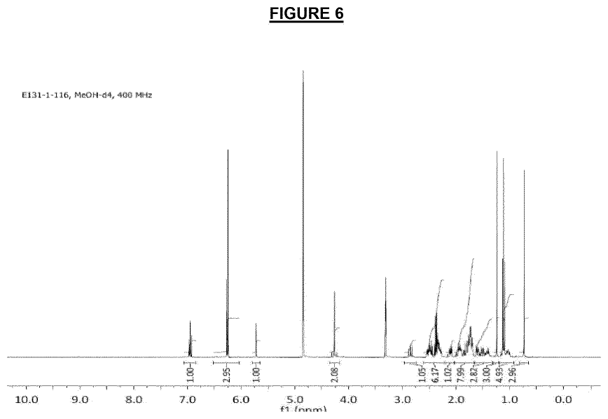

According to a preferred embodiment, said crystalline form D has a proton NMR spectrum as shown in FIG. 6.

Preferably, this crystalline form D is obtained by crystallization of cortexolone 17α-propionate with resorcinol in a 1:1 molar ratio in ethyl acetate mixed with an antisolvent, more preferably heptane.

As is well known to those skilled in the art, a crystalline form can be obtained by crystallization of a chemical compound in a specific solvent or mixture of solvents, possibly introducing a seed, i.e. a small quantity of crystallized compound, into the reaction mixture.

Preferably, said crystalline form D is obtained by seeding the same.

According to a further preferred embodiment, said cocrystal comprises cortexolone 17α-propionate and m-cresol in a 1:1 molar ratio in crystalline form F.

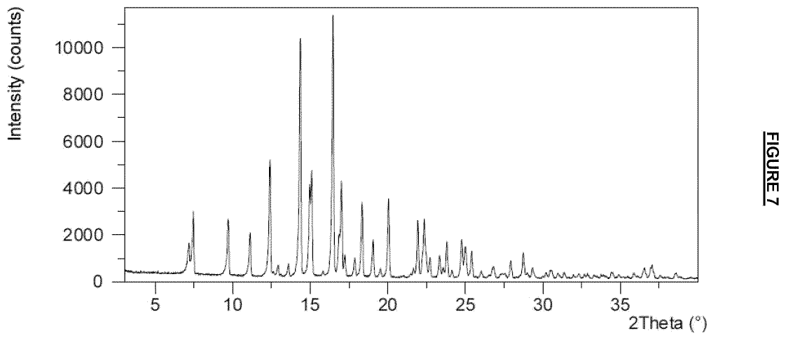

More preferably, such crystalline form F is characterized by an XRPD spectrum having the following characteristic peaks at approximately 7.2; 7.5; 9.7; 11.1; 12.4; 14.4; 15.0; 15.1; 16.5; 17.0; 18.3; 20.0; 21.9; 22.3±0.2° 2θ or as shown in FIG. 7.

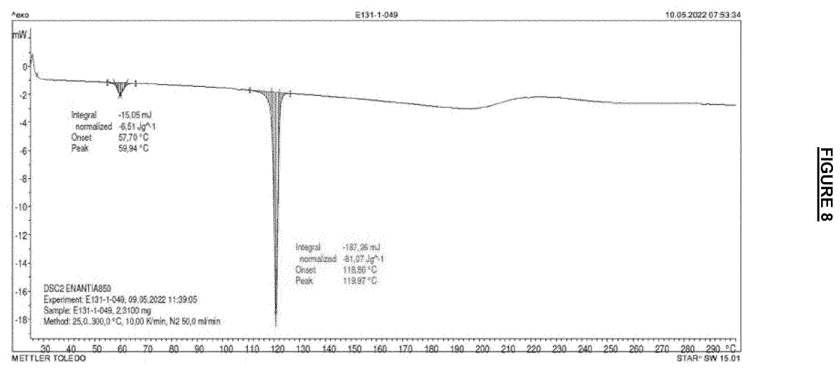

According to a preferred embodiment, said crystalline form F has a melting point at approximately 119° C. and a DSC thermogram as shown in FIG. 8.

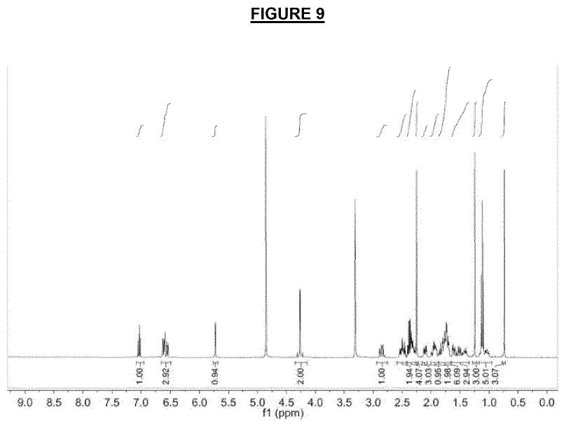

According to a preferred embodiment, said crystalline form F has a proton NMR spectrum as reported in FIG. 9.

Preferably, this crystalline form F is obtained by suspending cortexolone 17α-propionate in crystalline form III with m-cresol in a 1:1 molar ratio in diisopropyl ether.

According to a further preferred embodiment, said cocrystal comprises cortexolone 17α-propionate and m-cresol in a 1:1 molar ratio in crystalline form G. This crystalline form represents a conformation polymorph with respect to the crystalline form F reported above.

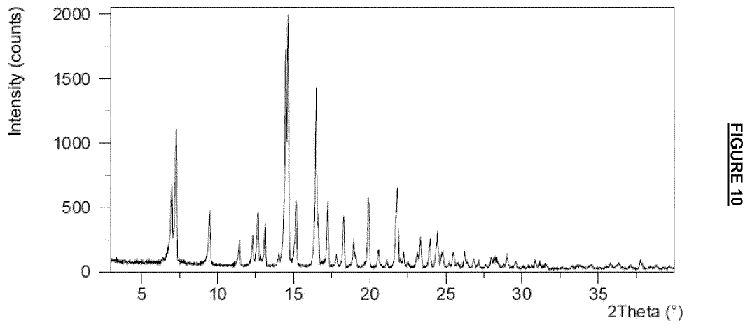

More preferably, such crystalline form G is characterized by an XRPD spectrum having the following characteristic peaks at approximately 7.0; 7.3; 9.5; 12.7; 14.5; 14.7; 15.2; 16.5; 16.6; 17.2; 18.3; 19.9; 21.8±0.2° 2θ or as shown in FIG. 10.

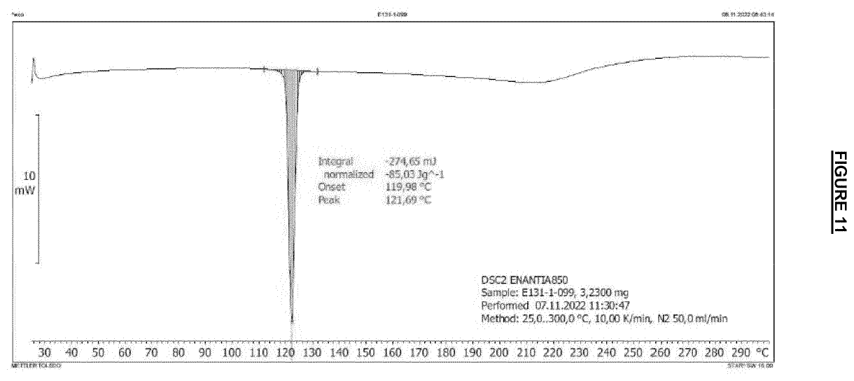

According to a preferred embodiment, said crystalline form G has a melting point at approximately 119° C. and a DSC thermogram as shown in FIG. 11.

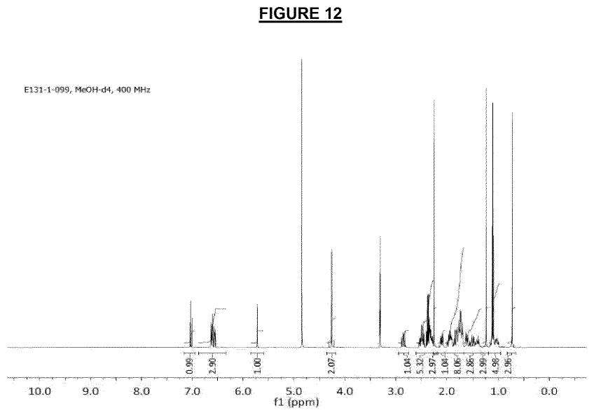

According to a preferred embodiment, said crystalline form G has a proton NMR spectrum as shown in FIG. 12.

Preferably, this crystalline form G is obtained by crystallization of cortexolone 17α-propionate with m-cresol in a 1:1 molar ratio in methyl isobutyl ketone mixed with an antisolvent, more preferably heptane.

According to a further preferred embodiment, said cocrystal comprises cortexolone 17α-propionate and 3,4-xylenol in a 1:1 molar ratio in crystalline form H.

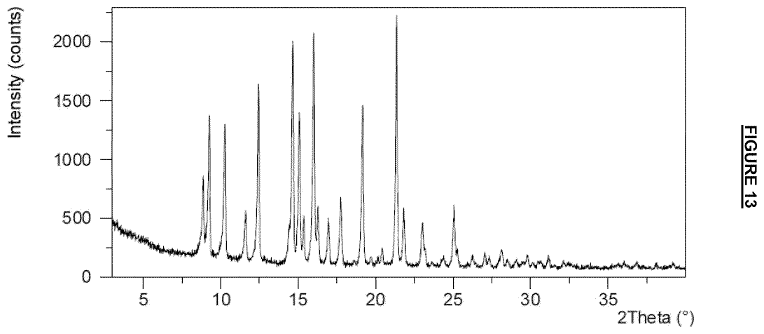

More preferably, such crystalline form H is characterized by an XRPD spectrum having the following characteristic peaks at approximately 8.9; 9.3; 10.3; 12.4; 14.7; 15.1; 16.0; 16.3; 17.7; 19.2; 21.4; 25.1±0.2° 2θ or as shown in FIG. 13.

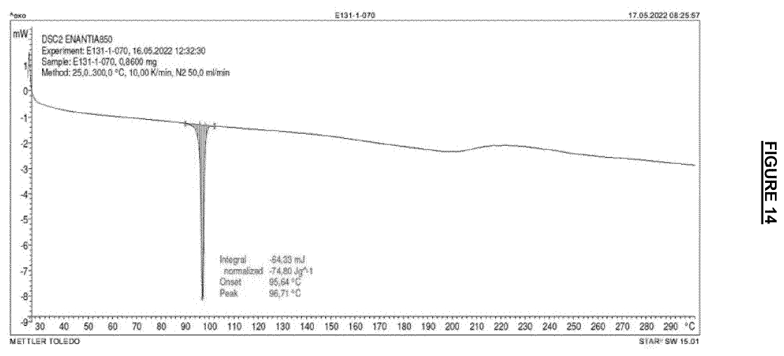

According to a preferred embodiment, said crystalline form H has a melting point at approximately 95° C. and a DSC thermogram as shown in FIG. 14.

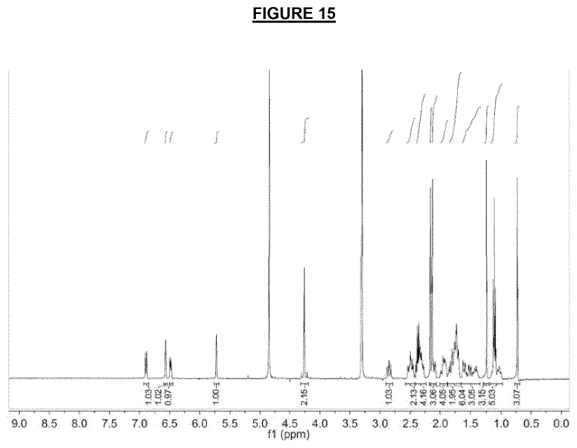

According to a preferred embodiment, said crystalline form H has a proton NMR spectrum as reported in FIG. 15.

Preferably, such crystalline form H is obtained by suspending cortexolone 17α-propionate in crystalline form III with 3,4-xylenol in a 1:1 molar ratio in cyclohexane.

According to a further preferred embodiment, said cocrystal comprises cortexolone 17α-propionate and o-cresol in a 1:1 molar ratio in crystalline form I.

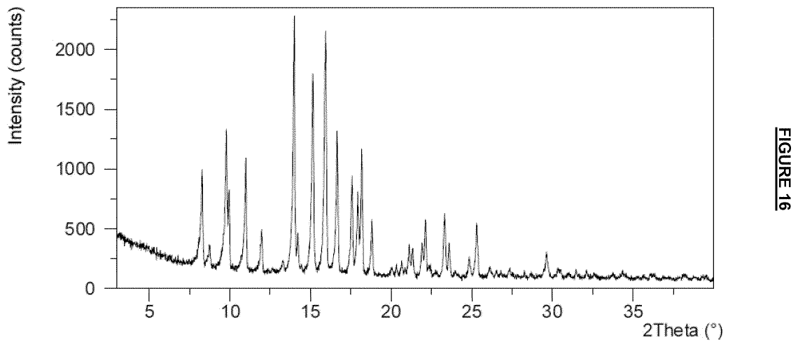

More preferably, such crystalline form I is characterized by an XRPD spectrum having the following characteristic peaks at approximately 8.3; 9.8; 9.9; 11.0; 14.0; 15.2; 15.9; 16.7; 17.6; 17.9; 18.2; 22.1; 23.3; 25.3±0.2° 2θ or as shown in FIG. 16.

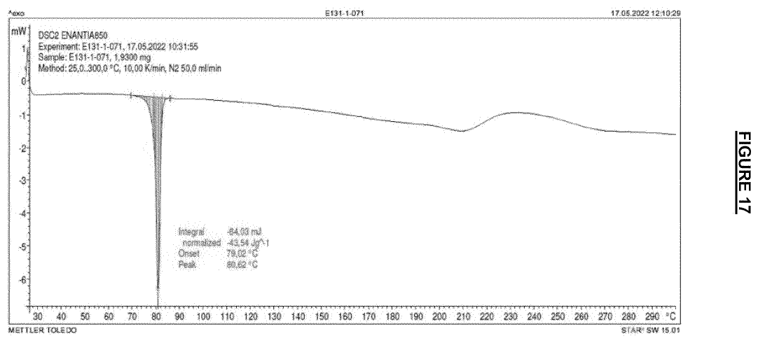

According to a preferred embodiment, said crystalline form I has a melting point at approximately 79° C. and a DSC thermogram as shown in FIG. 17.

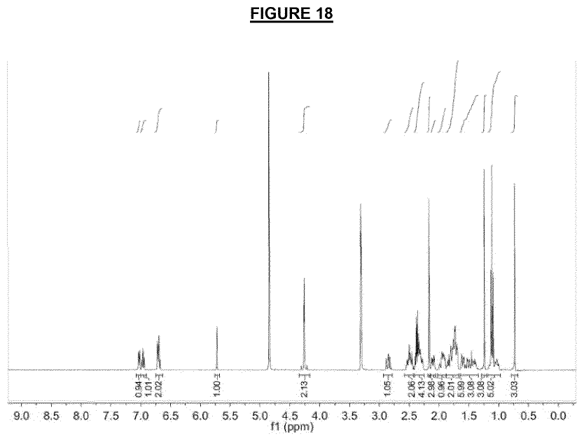

According to a preferred embodiment, said crystalline form I has a proton NMR spectrum as shown in FIG. 18.

Preferably, such crystalline form I is obtained by suspending cortexolone 17α-propionate in crystalline form III with o-cresol in a 1:1 molar ratio in cyclohexane.

According to a further preferred embodiment, said cocrystal comprises cortexolone 17α-propionate and resorcinol in a 1:1 molar ratio in crystalline form J.

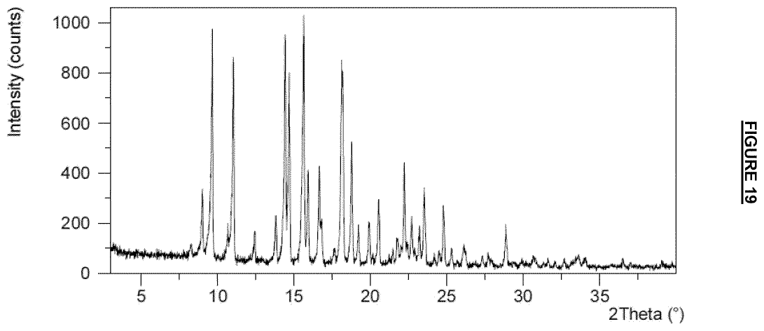

More preferably, such crystalline form J is characterized by an XRPD spectrum having the following characteristic peaks at approximately 9.0; 9.7; 11.0; 14.4; 14.7; 15.6; 15.9; 16.7; 18.1; 18.2; 18.8; 20.5; 22.2; 22.7; 23.5; 24.8±0.2° 2θ or as shown in FIG. 19.

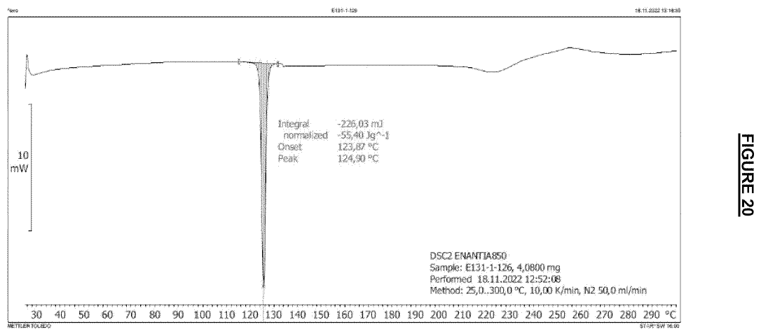

According to a preferred embodiment, said crystalline form J has a melting point at approximately 123° C. and a DSC thermogram as shown in FIG. 20.

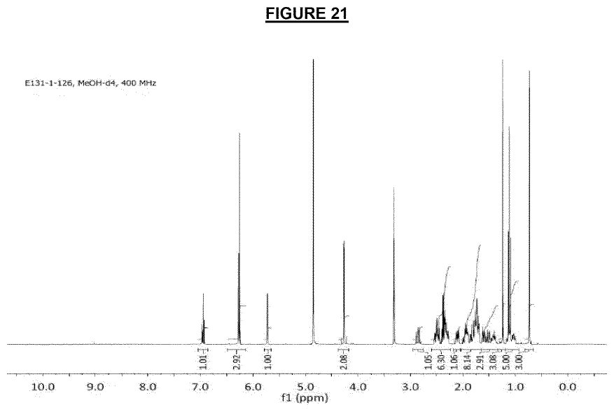

According to a preferred embodiment, said crystalline form J has a proton NMR spectrum as reported in FIG. 21.

Preferably, this crystalline form J is obtained by crystallization of cortexolone 17α-propionate with resorcinol in a 1:2 molar ratio in ethyl acetate mixed with an antisolvent, more preferably heptane.

Preferably, said crystalline form J is obtained by seeding the same.

A further object of the present invention is a pharmaceutical composition comprising a cocrystal of cortexolone 17α-propionate and at least one pharmaceutically acceptable excipient.

From preliminary stability data, two preferred forms were selected.

Preferably, said cortexolone 17α-propionate cocrystal is one of the crystalline forms reported above.

More preferably, said cortexolone 17α-propionate cocrystal is selected between the crystalline form G and the crystalline form J.

The cocrystals of the present invention display several advantages over the crystalline forms disclosed in the art, such as a reduced wettability making them non-hygroscopic and different solubility, thus leading to a different bioavailability. These data showed that cocrystals can be used in new formulations.

The present invention will now be illustrated by means of some examples, which should not be seen as limiting the scope of the invention.

### Experimental Part

Diffraction measurements were performed under ambient conditions on a PANalytical X'Pert PRO θ-θ diffractometer of 240 mm radius in reflection geometry, equipped with CuKα radiation and a PIXcel detector, operated at 45 kV and 40 mA. Each sample was mounted on a zero-bottom silicone holder and allowed to rotate at 0.25 rpm during data collection. The angular measurement range was 3.0-40.0° (2θ) with a step size of 0.013°. The scanning speed was 0.082 (40.80 s/step).

Nuclear magnetic resonance (proton NMR) analyzes were recorded in deuterated methanol in a Varian Mercury 400 MHz spectrometer, equipped with a 5 mm ATB 1H/19F/X broadband probe. Spectra were acquired by dissolving 5-10 mg of sample in 0.7 mL of MeOD-d4.

DSC analyzes were recorded with a Mettler Toledo DSC2. The sample was weighed into a 40 μL aluminum crucible with a pinhole lid and heated from 25 to 300° C. at a rate of 10° C./min, under nitrogen (50 mL/min).

Wettability analysis has been carried out by a contact angle meter (Kyowa Interface Science, made in Japan, model: DMe-211Plus) with FAMAS software for data processing.

### Example 1

### Preparation of Crystalline Form D

To a mechanically stirred tube containing cortexolone 17α-propionate (25 mg, 0.062 mmol) and resorcinol (14 mg, 0.127 mmol, 2 eq), isopropyl ether (0.25 ml, 10V) was added and complete solution was observed at room temperature. The solution was stirred at room temperature overnight. The solid precipitate was isolated by filtration and dried under vacuum (approximately 3 mbar) at room temperature overnight. Form D was obtained, contaminated by cortexolone 17α-propionate Form III, according to XRD analysis.

### Example 2

### Preparation of Crystalline Form D

To a mechanically stirred round bottom flask containing cortexolone 17α-propionate (4.00 g, 9.94 mmol) and resorcinol (1.10 g, 9.99 mmol, 1 eq.), ethyl acetate (8 mL, 2 V) was added and complete dissolution was observed at room temperature. The solution was seeded with Form D and heptane (32 mL, 8 V) was added dropwise (for approximately 10-15 minutes) before stirring at room temperature for 1 hour and at 0-5° C. for 30 minutes. The resulting solid was filtered through a sintered funnel (porous #3), washed with heptane (10 mL, 2.5 V), and dried under vacuum (approximately 3 mbar) at room temperature overnight. Form D was obtained as a whitish solid according to XRPD (4.55 g, crystallization yield=89%).

### Example 3

### Preparation of Crystalline Form G

To a mechanically stirred round bottom flask containing cortexolone 17α-propionate (5.00 g, 12.4 mmol) and m-cresol (1.3 mL, 12.4 mmol, 1 eq.), methyl isobutyl ketone (10 mL, 2 V) was added and complete dissolution was observed at room temperature. Then, the solution was heated to 80° C. (internal temperature) and heptane (71.5 mL, 14.3 V) was added dropwise (in approximately 30 minutes). Instead of solid precipitation, oil formation was observed at the end of heptane addition. The mixture was seeded with Form G at 80° C. and stirred at this temperature for 1 hour, leading to crystallization of the product. After cooling to room temperature and stirring at this temperature for 2 h, the resulting suspension was filtered through a sintered funnel (porous #3), washed with heptane (10 ml, 2 V) and dried under vacuum (about 3 mbar) at room temperature overnight. Form G was obtained as a whitish solid according to XRPD (3.90 g, crystallization yield=32%).

### Example 4

### Preparation of Crystalline Form J

To a mechanically stirred test tube containing Cortexolone 17α-propionate (50 mg, 0.124 mmol) and Resorcinol (27 mg, 0.245 mmol, 2 eq), ethyl acetate (0.25 ml, 5V) was added and complete solution was observed at room temperature. A Form D seed crystal was added to the solution and heptane (1 ml, 20V) was added dropwise. A light precipitate was observed at room temperature, and a Form D seed crystal was added again and stirred at 0-5° C. After 2 days, a good suspension was obtained. The solid precipitate was isolated by filtration and dried under vacuum (about 3 mbar) at room temperature overnight, obtaining Form J.

### Example 5

### Preparation of Crystalline Form J

To a mechanically stirred round bottom flask containing cortexolone 17α-propionate (5.00 g, 12.4 mmol) and resorcinol (1.37 g, 12.4 mmol, 1 eq.), ethyl acetate (10 mL, 2 V) was added and complete dissolution was observed at room temperature. The solution was seeded with Form J and heptane (40 mL, 8 V) was added dropwise (in approximately 10-15 minutes). After 1 hour of stirring at room temperature, a second portion of resorcinol (274 mg, 2.49 mmol, 0.2 eq.) was added to the mixture before stirring at room temperature overnight. The solid was filtered through a sintered funnel (porous #3), washed with heptane (5 mL, 1 V), and dried under vacuum (approximately 3 mbar) at room temperature overnight. Form J was obtained as a whitish solid according to XRPD (5.52 g, crystallization yield=87%).

### Example 6

### Preparation of Crystalline Form B

To a magnetically stirred tube containing a mixture of cortexolone 17α-propionate Form III (100 mg, 0.248 mmol) and salicylic acid (69 mg, 0.497 mmol, 2 eq), methyl-t-butyl ether (0.5 ml, 0.5V) was added. A Form B seed crystal was added to the suspension thus obtained and stirred at 0-5° C. overnight. The suspension was filtered and washed with cold methyl-t-butyl ether (1×2V). It was dried under vacuum at room temperature overnight and analyzed by XRPD. Form B was obtained as a white solid (53 mg, 45% crystallization yield).

### Example 7

### Preparation of Crystalline Form F

To a magnetically stirred tube containing a mixture of cortexolone 17α-propionate Form III (50 mg, 0.124 mmol) and m-cresol (13 μl, 1 eq), isopropyl ether (0.5 ml, 10V) was added. A Form F seed crystal was added to the suspension thus obtained and stirred at room temperature overnight. The suspension was filtered and washed with isopropyl ether (2×2V). It was dried under vacuum at room temperature overnight and analyzed by XRPD. Form F was obtained as a white solid (39 mg, 62% crystallization yield).

### Example 8

### Preparation of Crystalline Form H

To a magnetically stirred tube containing a mixture of cortexolone 17α-propionate (50 mg, 0.124 mmol) and 3,4-xylenol (15 mg, 0.124 mmol, 1 eq), isobutyl acetate (0.25 ml, 5V) was added. A Form H seed crystal was added to the solution thus obtained and then stirred at room temperature overnight and then at 0-5° C. for 3 hours. The suspension was filtered and washed with cold isobutyl acetate (1×2V). It was dried under vacuum at room temperature overnight and analyzed by XRPD. Form H was obtained as a white solid (8.8 mg, 13% crystallization yield).

### Example 9

### Preparation of Crystalline Form I

To a magnetically stirred tube containing a mixture of cortexolone 17α-propionate (50 mg, 0.124 mmol) and o-cresol (13 μl, 0.124 mmol, 1 eq), cyclohexane (0.5 ml, 10V) was added. A Form I seed crystal was added to the suspension thus obtained and stirred at room temperature. The suspension was filtered and washed with cyclohexane (2×3V). It was dried under vacuum at room temperature overnight and analyzed by XRPD. Form I was obtained as a white solid (52 mg, 84% crystallization yield).

### Example 10

### Wettability

The contact angle of cocrystals (Form J and G) and Form III (reference) was evaluated at room temperature using ultrapure water (by MilliQ system). Cocrystals (Form J and G) and Form III were previously grounded with a mortar and pestle to limit the effect of particle sizes. Then, approximately 27 mg of each sample were collected on a glass substrate limited to a Teflon O-ring (6.0 mm diameter) and gently leveled by a spatula. The contact time between the drop and sample was set at 9 s and the aqueous falling volume was 2.0 μl. The results of the analysis are reported below in Table 1.

TABLE 1
Results of wettability analysis at
room temperature in ultrapure water

| | Contact angle (degrees) | | | Average |
|---|---|---|---|---|
| | Sample 1 | Sample 2 | Sample 3 | (degrees) |
| Cocrystal | 131.0 | 128.4 | 133.3 | 130.9 |
| form J | | | | |
| Cocrystal | 128.5 | 124.2 | 124.2 | 125.63 |
| form G | | | | |
| Crystalline | 76.8 | 75.0 | 81.2 | 77.67 |
| form III | | | | |

It can be appreciated that whereas crystalline form III of the prior art is hydrophilic (contact angle <90 degrees), the cocrystals of the present invention display a significantly higher contact angle and are thus hydrophobic (contact angle >90 degrees).

These cocrystals are therefore less prone to being hygroscopic with respect to the polymorph disclosed in the prior art.

### Solubility Tests

The kinetic solubility study was performed with cocrystals form J and G and crystalline form III (as reference), at pH 5.1 (acetate buffer) and 6.0 (phosphate buffer) at room temperature. Each sample was analyzed in duplicate.

Cocrystals form J and G as well as the crystalline form III were previously grounded with mortar and pestle to reduce the particle size effects. Then, 250 mg of each powder were resuspended in the corresponding aqueous buffer saline solution (37.5 ml, 150V) under the same stirring speed (mechanical stirring) and in the same type of flask (150 ml 2-neck round-bottomed flask); this procedure was performed for both buffer solutions. At scheduled timing (0.5 h, 1 h, 3 h, 5 h, 24 h), aliquots of approximately 3 ml were withdrawn and filtered. Mother liquors were analyzed in triplicate by HPLC, and their pH values checked by pH meter (914 meter/conductometer, Methron).

Cocrystal form J

TABLE 2
Results of cocrystal Form J solubility study at room temperature
in a pH 5.1 buffer solution (concentration and pH values).

| | | |
|---|---|---|
| 1st duplicate | 2nd duplicate | Average |

| | Conc | | Conc | | Conc |
|---|---|---|---|---|---|
| Time | (μg/mL) | pH | (μg/mL) | pH | (μg/mL) |

| | | | | | | |
|---|---|---|---|---|---|---|
| 30 | min | 8.6 | 5.08 | 8.61 | 5.10 | 8.61 |
| 1 | h | 28.06 | 5.16 | 29.26 | 5.14 | 28.66 |
| 3 | h | 10.29 | 5.12 | 10.19 | 5.14 | 10.24 |
| 5 | h | 15.45 | 5.13 | 15.48 | 5.13 | 15.47 |
| 24 | h | 10.76 | 5.06 | 10.69 | 5.13 | 10.73 |

TABLE 3
Results of cocrystal Form J solubility study at room temperature
in a pH 6.0 buffer solution (concentration and pH values)

| | | |
|---|---|---|
| 1st duplicate | 2nd duplicate | Average |

| | Conc | | Conc | | Conc |
|---|---|---|---|---|---|
| Time | (μg/mL) | pH | (μg/mL) | pH | (μg/mL) |

| | | | | | | |
|---|---|---|---|---|---|---|
| 30 | min | 16.1 | 6.01 | 17.12 | 6.04 | 17.01 |
| 1 | h | 4.29 | 6.02 | 4.26 | 6.02 | 4.27 |
| 3 | h | 3.27 | 5.99 | 3.29 | 6.01 | 3.31 |
| 5 | h | 1.53 | 5.99 | 1.52 | 5.99 | 1.54 |
| 24 | h | — | 5.99 | — | 6.07 | — |

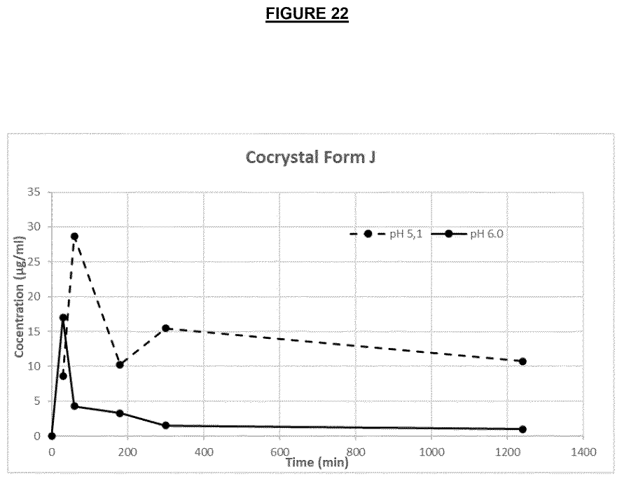

As it can be appreciated in FIG. 22, the dissolution of cocrystal Form J at pH 5.1 is very slow. The highest solubility was reached after 1 h (28.66±0.85 μg/mL). Then, a decrease of the solubility over time was observed. At pH 6.0 the highest solubility (17.01±0.15 μg/mL) was achieved in only 30 min, following a rapid decrease of solubility.

No significant variations were detected over time.

### Cocrystal Form G

TABLE 4
Results of cocrystal Form G solubility study at room temperature
in a pH 5.1 buffer solution (concentration and pH values)

| | | |
|---|---|---|
| 1st duplicate | 2nd duplicate | Average |

| | Conc | | Conc | | Conc |
|---|---|---|---|---|---|
| Time | (μg/mL) | pH | (μg/mL) | pH | (μg/mL) |

| | | | | | | |
|---|---|---|---|---|---|---|
| 30 | min | 2.37 | 5.15 | 2.32 | 5.17 | 2.35 |
| 1 | h | 6.72 | 5.12 | 6.77 | 5.14 | 6.75 |
| 3 | h | 1.29 | 5.11 | 1.34 | 5.15 | 1.32 |
| 5 | h | 1.17 | 5.14 | 1.16 | 5.17 | 1.16 |
| 24 | h | 4.09 | 5.13 | 4.06 | 5.16 | 4.79 |

TABLE 5
Results of cocrystal Form G solubility study at room temperature
in a pH 6.0 buffer solution (concentration and pH values)

| | | |
|---|---|---|
| 1st duplicate | 2nd duplicate | Average |

| | Conc | | Conc | | Conc |
|---|---|---|---|---|---|
| Time | (μg/mL) | pH | (μg/mL) | pH | (μg/mL) |

| | | | | | | |
|---|---|---|---|---|---|---|
| 30 | min | 1.78 | 6.08 | 1.78 | 6.09 | 1.78 |
| 1 | h | 4.20 | 6.10 | 3.98 | 6.07 | 4.09 |
| 3 | h | 0.95 | 6.12 | 0.96 | 6.09 | 0.95 |
| 5 | h | 1.53 | 6.09 | 1.52 | 6.04 | 1.52 |
| 24 | h | 1.92 | 6.0 | 1.96 | 6.0 | 1.94 |

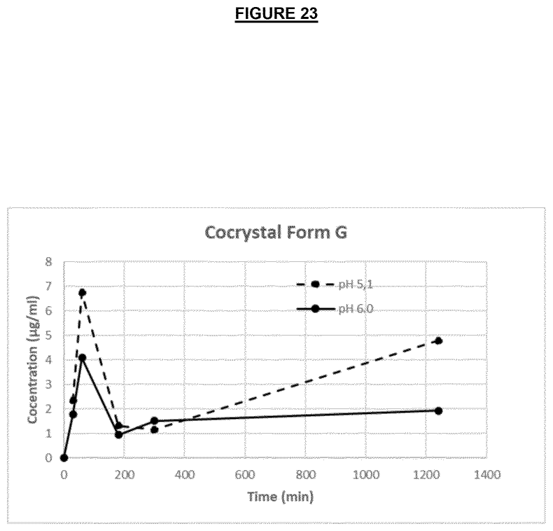

As it can be appreciated in FIG. 23, the dissolution of cocrystal Form G at pH 5.1 and pH 6.0 is very slow, the highest solubility values was achieved after 1 h at concentration of 6.75±0.04 μg/mL and 4.09±0.46 μg/mL, respectively. Afterwards, a decrease of the solubility over time was observed.

No significant pH variations were detected over time.

### Crystalline Form III (Prior Art)

TABLE 6
Results of crystalline form III solubility study at room temperature
in a pH 5.1 buffer solution (concentration and pH values)

| | | |
|---|---|---|
| 1st duplicate | 2nd duplicate | Average |

| | Conc | | Conc | | Conc |
|---|---|---|---|---|---|
| Time | (μg/mL) | pH | (μg/mL) | pH | (μg/mL) |

| | | | | | | |
|---|---|---|---|---|---|---|
| 30 | min | 18.07 | 5.19 | 18.12 | 5.19 | 18.07 |
| 1 | h | 10.02 | 5.23 | 10.0 | 5.21 | 10.01 |
| 3 | h | 16.96 | 5.22 | 16.82 | 5.19 | 16.89 |
| 5 | h | 14.68 | 5.22 | 15.02 | 5.21 | 14.77 |
| 24 | h | 2.59 | 5.16 | 2.60 | 5.20 | 2.60 |

TABLE 7
Results of crystalline form III solubility study at room temperature
in a pH 6.0 buffer solution (concentration and pH values)

| | | |
|---|---|---|
| 1st duplicate | 2nd duplicate | Average |

| | Conc | | Conc | | Conc |
|---|---|---|---|---|---|
| Time | (μg/mL) | pH | (μg/mL) | pH | (μg/mL) |

| | | | | | | |
|---|---|---|---|---|---|---|
| 30 | min | 4.38 | 6.02 | 4.37 | 6.05 | 4.38 |
| 1 | h | 3.58 | 6.05 | 3.56 | 6.05 | 3.57 |
| 3 | h | 11.15 | 5.98 | 10.97 | 5.98 | 11.06 |
| 5 | h | 1.92 | 6.0 | 1.92 | 6.0 | 1.92 |
| 24 | h | 4.02 | 6.02 | 4.03 | 6.02 | 4.02 |

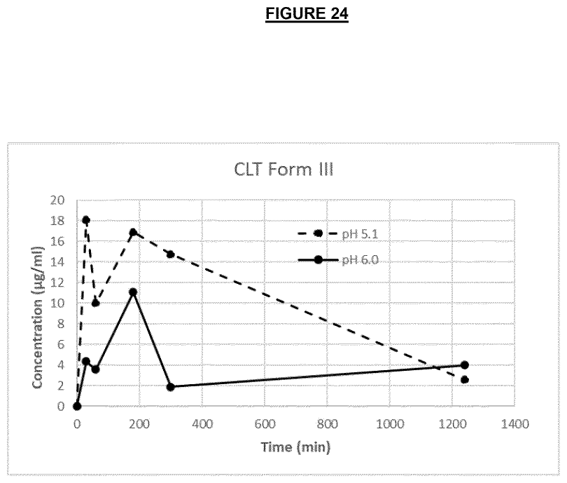

As it can be appreciated in FIG. 24, the dissolution of Form III at pH 5.1 is faster than the one at pH 6.0 reaching the highest solubility (18.07±0.06 μg/mL) after only 30 min. Then a decrease of the solubility with time was highlighted. At the pH 6.0 the highest solubility (15.97±0.04 μg/mL) was achieved after 30 min, following the decrease of solubility.

No significant pH variations were detected over time.

Based on the results, it is possible to conclude that the cocrystals of the present invention have higher hydrophobic properties, showed by high contact angle, and different solubility profiles at the tested pHs (5.1 and 6.0) with respect to crystalline form III disclosed in the prior art.

### Morphological Analysis

The morphology of the samples (Form J, Form G and Form III) was analyzed by scanning electron microscopy (SEM), Zeiss EVO MA10 apparatus (Carl Zeiss, Oberkochen, Germany). The samples were fixed on carbon double-sided tape and metalized with gold. Scanning electron photographs were taken at accelerated voltages (20 kV), in high vacuum and at room temperature. The samples were observed at different magnifications (500×, 1.0 KX and 2.0 KX).

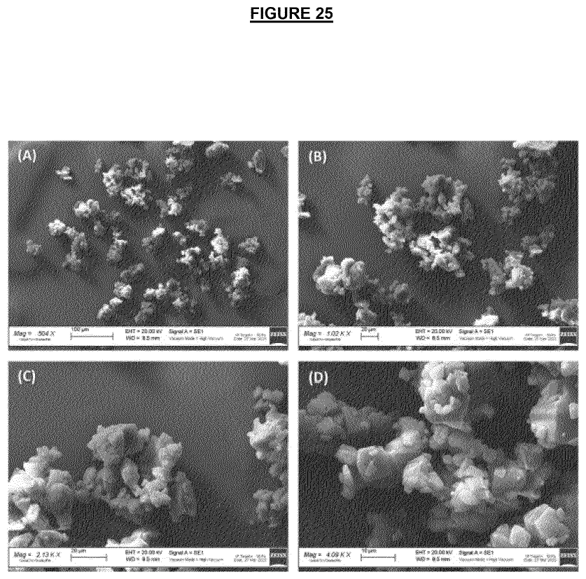

FIG. 25 shows SEM images of cocrystal form J, displaying irregular-shaped plates that are generally aggregated into large particles (>10 μm). At high magnification (Image D) cocrystal Form J shows clearly spherical/block-shaped particles.

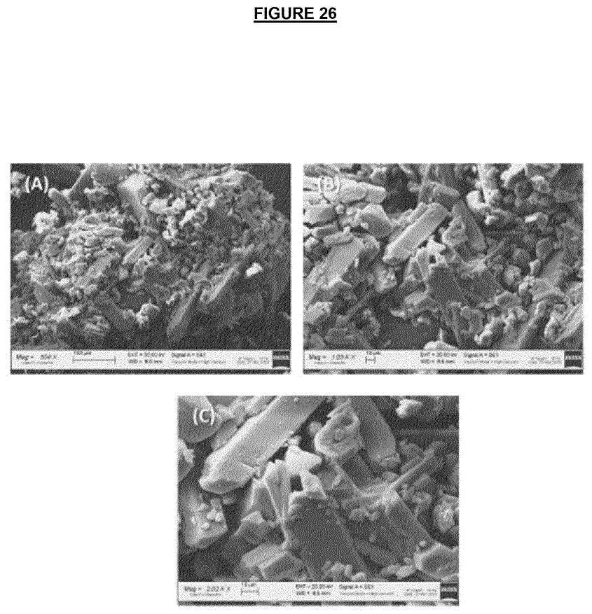

Representative SEMs of cocrystal form G, shown in FIG. 26, indicate large aggregates with needle-like morphology of cocrystal. The particle size of these crystals is significantly larger than the cocrystal Form J (>100 μm).

FIGS. 25 and 26 show that the coformer used for the co-crystallization influenced the final crystal morphology, particle size and particle size distribution.

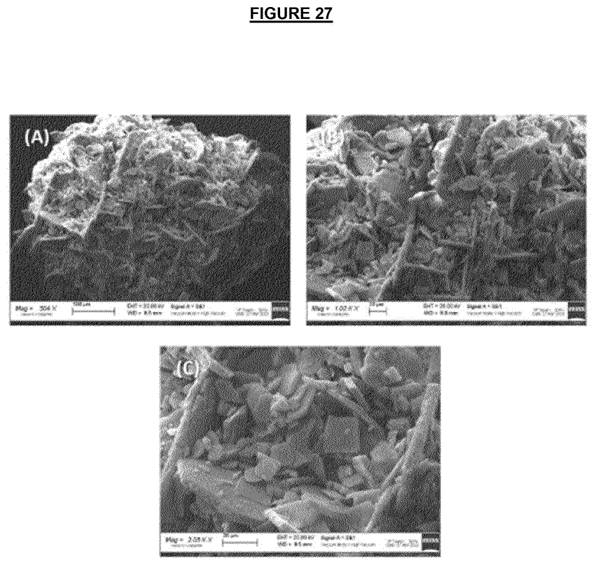

Crystalline form III exhibit irregular plate-shaped plates aggregated into dense large aggregates in FIG. 27.

1. A cocrystal of cortexolone 17α-propionate with a coformer.

2. The cocrystal according to claim 1, wherein said coformer is a substituted aromatic compound.

3. The cocrystal according to claim 1, wherein said cocrystal is cortexolone 17α-propionate with salicylic acid in a 2:1 molar ratio in crystalline form B, characterized by the fact that the XRPD spectrum has the following characteristic peaks at approximately 5.4; 13.7; 14.1; 14.4; 14.7; 16.2; 22.6±0.2° 2θ or as reported in FIG. 1 and/or a DSC thermogram as reported in FIG. 2 and/or a proton NMR spectrum as reported in FIG. 3.

4. The cocrystal according to claim 1, wherein said cocrystal is cortexolone 17α-propionate with resorcinol in a 1:1 molar ratio in crystalline form D, characterized by the fact that the XRPD spectrum has the following characteristic peaks at approximately 10.6; 12.1; 14.6; 14.8; 16.0; 19.7; 21.3±0.2° 2θ or as reported in FIG. 4 and/or a melting point at about 109° C. and a DSC thermogram as reported in FIG. 5 and/or a proton NMR spectrum as reported in FIG. 6.

5. The cocrystal according to claim 1, wherein said cocrystal is cortexolone 17α-propionate with m-cresol in a 1:1 molar ratio in crystalline form F, characterized by the fact that the XRPD spectrum has the following characteristic peaks at approximately 7.2; 7.5; 9.7; 11.1; 12.4; 14.4; 15.0; 15.1; 16.5; 17.0; 18.3; 20.0; 21.9; 22.3±0.2° 2θ or as reported in FIG. 7 and/or a melting point at about 119° C. and a DSC thermogram as reported in FIG. 8 and/or a proton NMR spectrum as reported in FIG. 9.

6. The cocrystal according to claim 1, wherein said cocrystal is cortexolone 17α-propionate with m-cresol in a 1:1 molar ratio in crystalline form G, characterized by the fact that the XRPD spectrum has the following characteristic peaks at approximately 7.0; 7.3; 9.5; 12.7; 14.5; 14.7; 15.2; 16.5; 16.6; 17.2; 18.3; 19.9; 21.8±0.2° 2θ or as reported in FIG. 10 and/or a melting point at about 119° C. and a DSC thermogram as reported in FIG. 11 and/or a proton NMR spectrum as reported in FIG. 12.

7. The cocrystal according to claim 1, wherein said cocrystal is cortexolone 17α-propionate with 3,4-xylenol in a 1:1 molar ratio in crystalline form H, characterized by the fact that the XRPD spectrum has the following characteristic peaks at approximately 8.9; 9.3; 10.3; 12.4; 14.7; 15.1; 16.0; 16.3; 17.7; 19.2; 21.4; 25.1±0.2° 2θ or as reported in FIG. 13 and/or a melting point at about 95° C. and a DSC thermogram as reported in FIG. 14 and/or a proton NMR spectrum as reported in FIG. 15.

8. The cocrystal according to claim 1, wherein said cocrystal is cortexolone 17α-propionate with o-cresol in a 1:1 molar ratio in crystalline form I, characterized in that the XRPD spectrum has the following characteristic peaks at approximately 8.3; 9.8; 9.9; 11.0; 14.0; 15.2; 15.9; 16.7; 17.6; 17.9; 18.2; 22.1; 23.3; 25.3±0.2° 2θ or as reported in FIG. 16, and/or a melting point at about 79° C. and a DSC thermogram as reported in FIG. 17 and/or a proton NMR spectrum as reported in FIG. 18.

9. The cocrystal according to claim 1, wherein said cocrystal is cortexolone 17α-propionate with resorcinol in a 1:1 molar ratio in crystalline form J, characterized by the fact that the XRPD spectrum has the following characteristic peaks at approximately 9.0; 9.7; 11.0; 14.4; 14.7; 15.6; 15.9; 16.7; 18.1; 18.2; 18.8; 20.5; 22.2; 22.7; 23.5; 24.8±0.2° 2θ or as reported in FIG. 19 and/or a melting point at about 123° C. and a DSC thermogram as reported in FIG. 20 and/or a proton NMR spectrum as reported in FIG. 21.

10. A pharmaceutical composition comprising a cocrystal of cortexolone 17α-propionate and at least a pharmaceutically acceptable excipient.

11. The pharmaceutical composition according to claim 10, wherein said cocrystal is in crystalline form G or in crystalline form J.

12. The cocrystal according to claim 2, wherein said substituted aromatic compound is selected from a group consisting of salicylic acid, resorcinol, m-cresol, o-cresol, and 3.4-xylenol.
